(19) United States
(12) Patent Application Publication
(10) Pub. No.: US 2005/0054037 A1
(43) Pub. Date: Mar. 10, 2005

(54) Ferroportin-1 mutant

(76) Inventors: Daniel Wallace, Queensland (AU); Katherine Arden, Queensland (AU); Palle Pedersen, Naestved (DK); Nathan Subramaniam, Queensland (AU)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/490,795

(22) PCT Filed: Oct. 1, 2002
(86) PCT No.: PCT/AU02/01335

(30) Foreign Application Priority Data
Oct. 1, 2001 (AU) PR7992

(51) Int. Cl. C07H 21/04; C12P 21/04; C07K 16/18; C07K 14/795

(52) U.S. Cl. 435/69.1; 435/320.1; 435/325; 530/400; 530/388.25; 536/23.5

(57) ABSTRACT

A mutant, human ferroportin-1 protein and encoding nucleic acid are provided. The mutant ferroportin-1 protein has a deletion of valine 162 compared to wild-type ferroportin-1 protein. The mutant protein and nucleic acid may be useful in detection of a predisposition to iron overload disorders such as haemochromatosis. Furthermore, it is proposed that the valine 162 deletion is a loss-of-function mutation that may underlie iron overload disorders such as haemochromatosis. Therefore, methods of both diagnosis and treatment of haemochromatosis are provided.

### FIELD OF THE INVENTION

THIS INVENTION relates to a mutant ferroportin-1 protein and encoding nucleic acid. More particularly, this invention relates to a loss-of-function mutant ferroportin-1 protein that underlies hereditary iron overload diseases such as haemochromatosis. This invention also provides methods of detecting mutant ferroportin-1 protein and encoding nucleic acid for the purposes of haemochromatosis diagnosis.

### BACKGROUND OF THE INVENTION

The most common form of hereditary iron overload is caused by mutations in the HFE gene (HFE-related haemochromatosis). This is an autosomal recessive disorder affecting approximately 1 in 200 people of northern European origin.¹ Progressive accumulation of iron can lead to tissue damage including cirrhosis, diabetes mellitus, arthropathy, cardiomyopathy, endocrine abnormalities and hepatocellular carcinoma.² The gene responsible (HFE) was identified in 1996 and homozygosity for a missense mutation C282Y was found to be responsible for the majority of cases.³ Another missense mutation H63D was also identified. H63D appears to have a much lower penetrance than C282Y. However, some compound heterozygotes for C282Y and H63D can develop iron overload.³

Other non-HFE related forms of iron overload have been described particularly in Southern Europe, where homozygosity for C282Y among haemochromatosis patients is less common.⁴ These include juvenile haemochromatosis (HFE2) and HFE3, which is caused by mutations in the transferrin receptor 2 (TfR2) gene. Juvenile haemochromatosis is inherited as an autosomal recessive trait. It is more severe than HFE related haemochromatosis. Onset usually occurs in the second to third decade of life. Cardiomyopathy and hypogonadism are common clinical findings and can lead to premature death if untreated.⁵ The HFE2 locus maps to chromosome 1q, however, the gene responsible has not yet been identified. Recently mutations in TfR2 have been implicated in a new form of haemochromatosis (HFE3), which is inherited in an autosomal recessive pattern and maps to chromosome 7q22.^{7,8} The clinical phenotype is variable among patients with TfR2 mutations. However, phenotypically the disease appears to be similar to HFE related haemochromatosis.⁸

Haemochromatosis families with apparent autosomal dominant inheritance have been reported.⁹⁻¹¹ Recently a new locus for an autosomal dominant form of haemochromatosis (HFE4) was identified on chromosome 2q32. Two missense mutations in the SLC11A3 gene, which maps to this region were detected in two families with autosomal dominant haemochromatosis from the Netherlands and Italy.^{12,13} The SLC11A3 gene, also known as ferroportin-1, REG1 and MTP1¹⁴⁻¹⁶ encodes a multiple transmembrane domain protein responsible for iron export from cells. Both gain of function and loss of function of ferroportin-1 were proposed to result in iron overload in these families.^{12,13} However, the authors were not able to distinguish whether gain of function or loss of function resulted in iron overload. In another recent report a mutation in the iron responsive element of H ferritin mRNA was shown to cause autosomal dominant haemochromatosis in a Japanese family.¹⁷

### OBJECT OF THE INVENTION

The present inventors have identified a new mutant form of ferroportin-1 protein and encoding mutant allele.

It is therefore an object of the invention to provide a loss-of-function mutant ferroportin-1 protein, an encoding nucleic acid and methods of using same for mutation detection of hereditary iron overload disease diagnosis.

### SUMMARY OF THE INVENTION

In a first aspect, the invention broadly relates to an isolated mutant ferroportin-1 protein.

Preferably, the mutant ferroportin-1 protein is a loss-of-function mutant.

Preferably, said ferroportin-1 protein mutant displays reduced iron-binding and/or iron transport activity compared to a corresponding wild-type ferroportin-1 protein.

Preferably, said isolated ferroportin-1 protein mutant has one or more amino acid deletions or non-conservative substitutions compared to a corresponding wild-type ferroportin-1 protein.

More preferably, said isolated ferroportin-1 protein mutant has a deletion or non-conservative substitution of a valine residue selected from the group consisting of: valine 160, valine 161 and valine 162.

Even more preferably, said isolated ferroportin-1 protein mutant has a deletion of a valine residue selected from the group consisting of: valine 160, valine 161 and valine 162.

In a preferred embodiment, said isolated ferroportin-1 protein mutant has a deletion of a valine 162.

In a particularly preferred embodiment, the ferroportin-1 mutant has the amino acid sequence of SEQ ID NO: 2.

This aspect of the invention also includes fragments of the isolated mutant ferroportin-1 protein that have a deletion of valine 160, 161 or 162.

In a second aspect, the invention provides an isolated nucleic acid which encodes the ferroportin-1 mutant of the first aspect.

Preferably, the isolated nucleic acid has a nucleotide sequence having one or more deleted or non-synonymous nucleotides that normally encodes a valine residue selected from the group consisting of: valine 160, valine 161 and valine 162.

More preferably, the isolated nucleic acid comprises a nucleotide sequence having nucleotides deleted that normally encode valine 162.

Isolated nucleic acids according to this aspect are referred to herein as mutant ferroportin-1 nucleic acids.

In a particularly preferred embodiment, the nucleic acid which encodes the ferroportin-1 mutant of the first aspect has the amino acid sequence of SEQ ID NO: 4.

This aspect of the invention extends to fragments of the isolated nucleic acid that have a deletion of one or more nucleotides that normally encode valine 160, 161 or 162.

Examples of fragments include the protein coding region of SEQ ID NO: 4 and a fragment corresponding to at least a portion of SEQ ID NO: 4 that corresponds to exon 5 of the humanferroportin-1 gene.

In a third aspect, the invention provides an antibody that binds a ferroportin-1 mutant protein of the first aspect, or a fragment thereof, but does not bind a corresponding wild-type ferroportin-1 protein or fragment thereof.

In a fourth aspect, the invention provides an expression construct comprising an isolated nucleic acid according to the second aspect operably-linked to one or more regulatory sequences in an expression vector.

In a fifth aspect, the invention provides a method of detecting a predisposition to an iron overload disorder, said method including the step of detecting a loss-of-function mutant ferroportin-1 protein according to the first aspect or an isolated mutant ferroportin-1 nucleic acid according to the second aspect, as an indication that an individual is predisposed to said iron overload disorder.

Preferably, the iron overload disorder is haemochromatosis.

Preferably, detection is performed by a PCR method.

Preferably, PCR amplification products corresponding to wild-type and loss-of-function ferroportin-1 nucleic acids respectively are identified by differential melting temperatures.

In a sixth aspect, the invention provides a method of treating an iron overload disorder, said method including the step of complementing a loss-of-function mutation in a ferroportin-1 protein or encoding nucleic acid in a mammal.

Preferably, the iron overload disorder is haemochromatosis.

Preferably, the mammal is a human.

In a seventh aspect, the invention provides a pharmaceutical composition for treating an iron overload disorder according to the sixth aspect of the invention.

Suitably, said pharmaceutical composition comprises a pharmaceutically acceptable carrier, diluent or excipient.

In one embodiment, said pharmaceutical composition comprises cells transformed, transfected or otherwise engineered to express a wild-type ferroportin-1 protein.

In another embodiment, said pharmaceutical composition comprises an expression construct comprising an isolated nucleic acid encoding a wild-type ferroportin-1 protein.

Suitably, said expression construct is a gene therapy construct suitable for administration to an mammal.

Preferably, the mammal is a human.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF THE FIGURES AND TABLES

Table 1. Details of affected family members.

Table 2. Primers used to amplify the 8 exons and splice site sequences of ferroportin-1 nucleic acid.

FIG. 1. Liver biopsy sections from affected family members stained with Perls' Prussian blue. (A) The proband (III:1), showing grade 4 iron accumulation at age 56 years. (B) The brother of the proband (III:3), showing grade 4 iron accumulation at age 73 years. (C) The son of the proband (IV:1), showing grade 3 iron accumulation at age 20 years. (D) The daughter of the proband (IV:2), showing grade 2 iron accumulation at age 19 years. In all patients iron is present most prominently in Kupffer cells. Iron overload is greater in the older patients, suggesting that iron accumulation is progressive with age.

FIG. 2. Family pedigree. The proband is indicated by an arrow. TS, transferrin saturation (%); SF, serum ferritin concentration (μg/L); Perls', Perls' stain grade (0-4); HIII, hepatic iron index.

FIG. 3. Mutation detection analysis by DNA sequencing and Denaturing HPLC. (A and B) Sequencing chromatograms of the forward sequence of exon 5, spanning the 485_{—}487delTTG mutation. (A) Unaffected family member III:2, showing normal sequence. (B) The proband, III:1, showing double sequence due to the deletion of TTG. (C-F) DHPLC analysis of exon 5. (C) Unaffected family member 111:2. (D) Control individual. (E) Affected family member 111:3. (F) Affected family member IV:2. Individuals heterozygous for the 485_{—}487delTTG mutation had 2 clear peaks corresponding to the homo- and hetero-duplex species (E and F). All un103 controls studied had a single clear peak, similar to the traces in C and D.

FIG. 4. Wild-type ferroportin-1 nucleic acid sequence (SEQ ID NO: 3). Bolded residues indicate the nucleotide sequence of exon 5.

FIG. 5. Wild-type ferroportin-1 protein sequence (SEQ ID NO: 1)

FIG. 6 Mutant Ferroportin-1 nucleic acid sequence (SEQ ID NO: 4) and encoded ferroportin-1 protein sequence (SEQ ID NO: 2) with deleted TTG trinucleotide and valine residue indicated by underlined dashes.

FIG. 7 Melting temperatures of the human wild-type genomic DNA and mutant allele DNA (TTG deletion) of the ferroportin-1 protein. The melt curve with the lower melting temperature denotes the mutant DNA and the melt curve with the higher melting temperature denotes the wild-type DNA.

### DETAILED DESCRIPTION OF THE INVENTION

The present invention arises from the inventors' discovery that a 3 base pair deletion in the ferroportin-1 gene (SLC11A3) is associated with autosomal dominant haemochromatosis in an Australian family. It is therefore proposed that this mutation which causes a deletion of a valine residue among residues 160-162 of the SLC11A3-encoded protein (ferroportin-1) is a loss of function mutation which results in impaired iron homeostasis leading to iron overload.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form.

The present invention provides an isolated ferroportin-1 mutant protein.

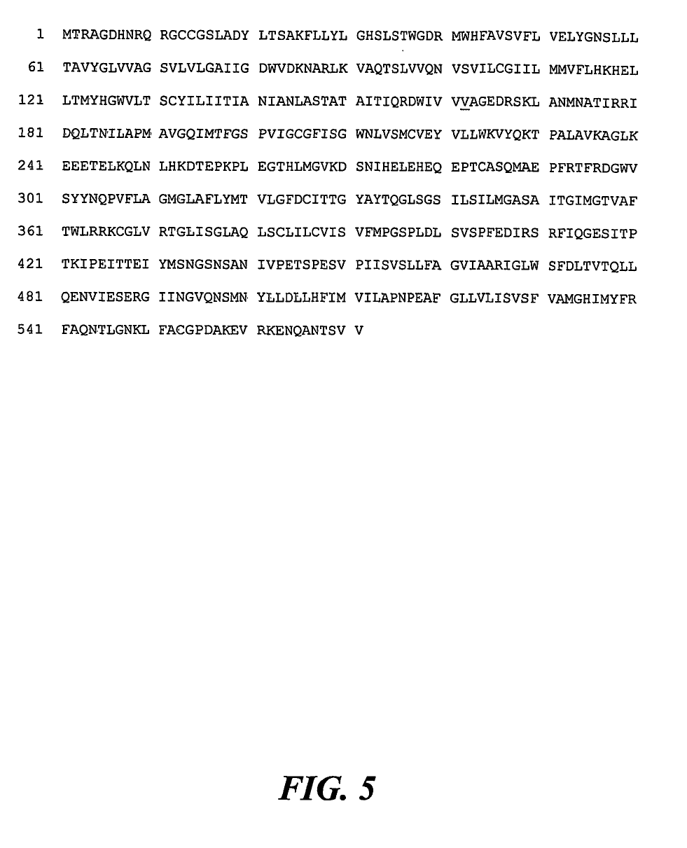

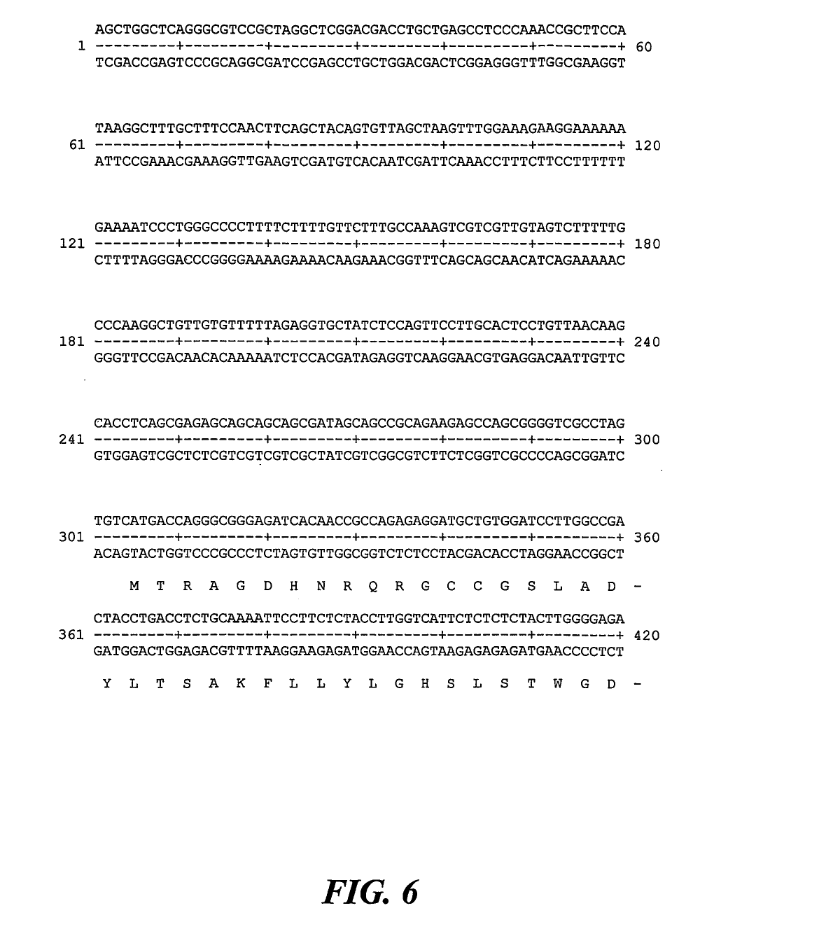

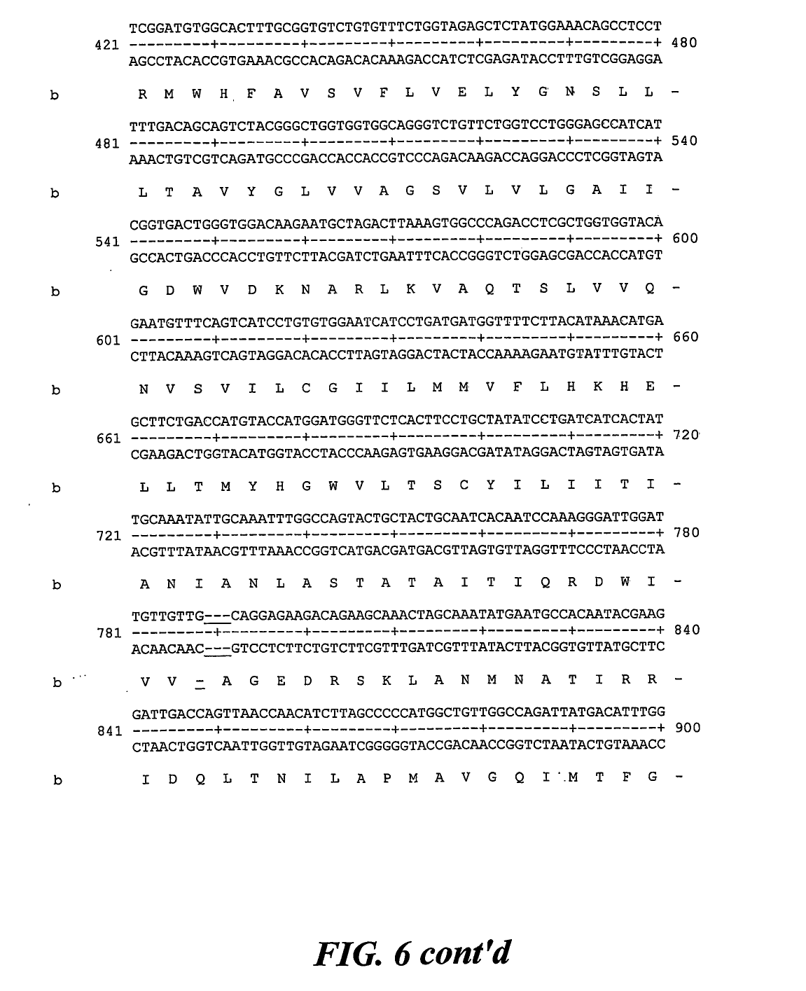

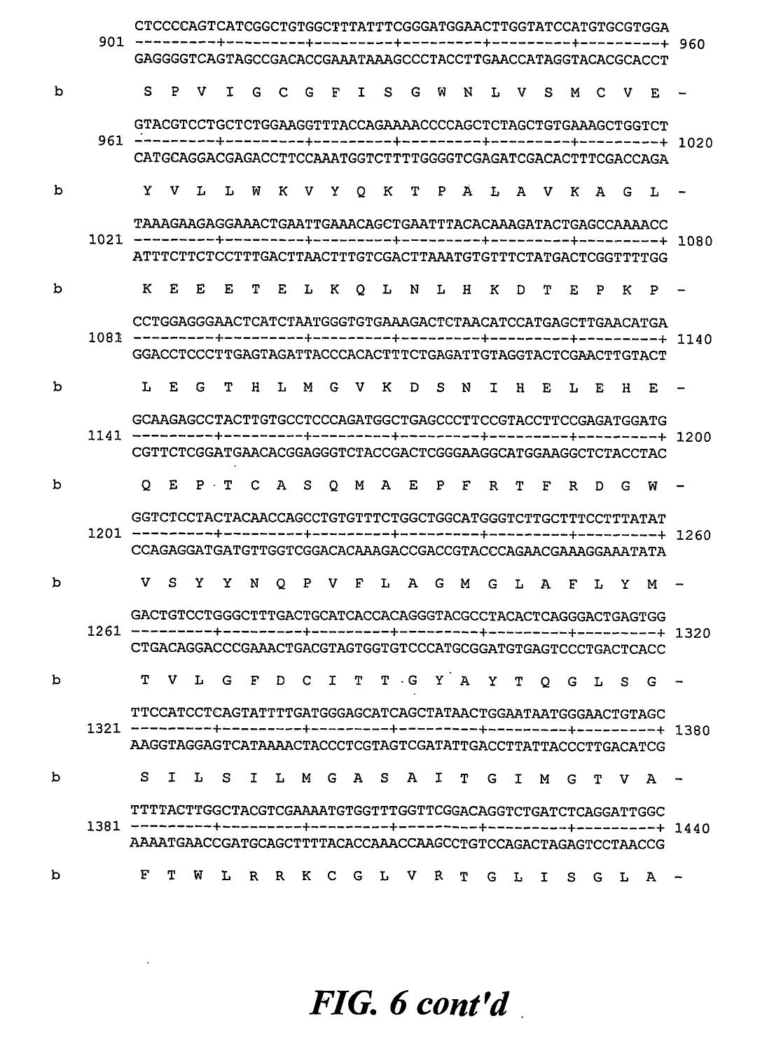

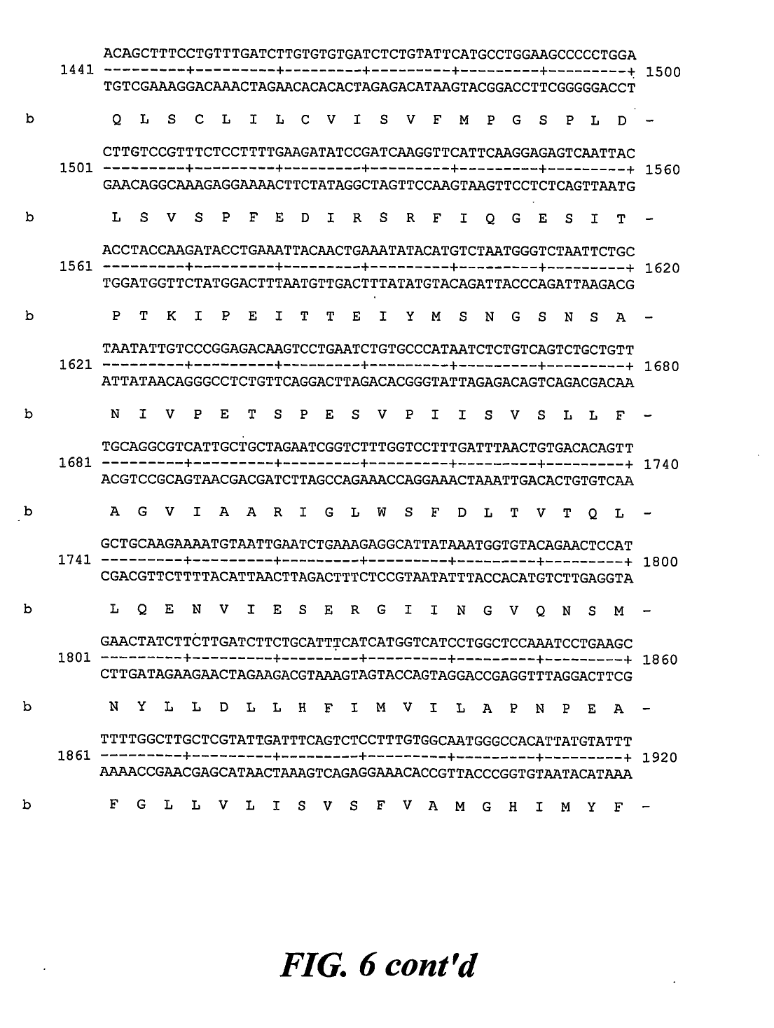

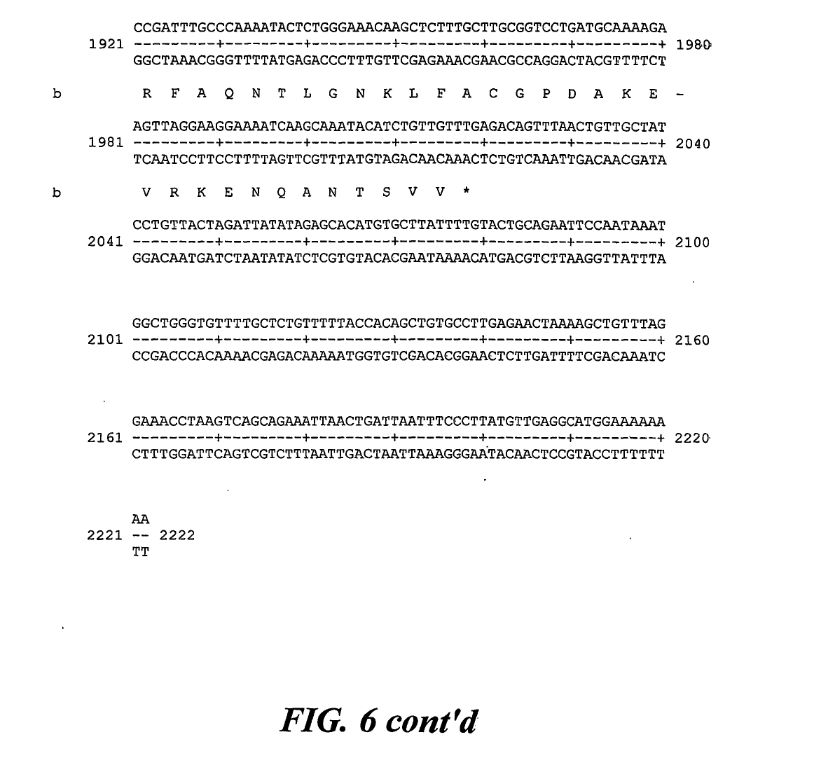

Deletion of the valine residue underlined in FIG. 6 produces a mutant ferroportin-1 protein sequence (SEQ ID NO: 2) of the invention compared to a corresponding wild-type ferroportin-1 protein sequence shown in FIG. 5 (SEQ ID NO: 1).

However, it will be appreciated that due to the presence of valine residues at positions 160, 161 and 162 of the wild-type sequence (SEQ ID NO:1), the assignation of any of these residue numbers to the deleted valine may be appropriate.

It is proposed by the present inventors that this is a loss-of-function mutant ferroportin-1 protein, although the present invention is not necessarily predicated or dependent upon this proposed functional effect of the mutation.

By “protein” is meant an amino acid polymer. The amino acids may be natural or non-natural amino acids, D- or L- amino acids or chemically-derivatized amino acids as are well understood in the art.

A “peptide” is a protein having less than fifty (50) amino acids.

A “polypeptide” is a protein having fifty (50) or more amino acids.

The invention also contemplates fragments of the isolated protein of the invention.

Suitably, said fragment comprises at least an amino acid sequence present in a ferroportin-1 mutant protein that is not present in a corresponding wild-type ferroportin-1 protein or vice versa.

In one embodiment, a “fragment” includes an amino acid sequence that constitutes less than 100%, but at least 10%, preferably at least 25%, more preferably at least 50% or even more preferably at least 75% of the isolated mutant ferroportin-1 protein (SEQ ID NO: 2).

For example, protein fragments have an amino acid sequence encoded by a portion of SEQ ID NO:4 corresponding to exon 5 of the human ferroportin-1 gene.

In another embodiment, a “fragment” is a small peptide, for example of at least 6, preferably at least 10 and more preferably at least 20 amino acids in length. Larger fragments comprising more than one peptide are also contemplated, and may be obtained through the application of standard recombinant nucleic acid techniques or synthesized using conventional liquid or solid phase synthesis techniques. Alternatively, peptides can be produced by digestion of a polypeptide of the invention with proteinases such as endoLys-C, endoArg-C, endoGlu-C and staphylococcus V8-protease. The digested fragments can be purified by, for example, high performance liquid chromatographic (HPLC) techniques.

The mutant ferroportin-1 protein of the invention has one or more amino acid deletions or non-conservative substitutions compared to a wild-type ferroportin-1 protein.

Generally, non-conservative substitutions which are likely to produce the greatest changes in protein structure and function are those in which (a) a hydrophilic residue (e.g., Ser or Thr) is substituted for, or by, a hydrophobic residue (e.g., Ala, Leu, Ile, Phe or Val); (b) a cysteine or proline is substituted for, or by, any other residue; (c) a residue having an electropositive side chain (e.g., Arg, His or Lys) is substituted for, or by, an electronegative residue (e.g., Glu or Asp) or (d) a residue having a bulky hydrophobic or aromatic side chain (e.g., Val, Ile, Phe or Trp) is substituted for, or by, one having a smaller side chain (e.g., Ala, Ser) or no side chain (e.g., Gly).

According to the present invention, non-conservative substitutions of Ala, Ser or Gly for any or all of valines 160, 161 or 162 of wild-type ferroportin-1 are potential examples of loss-of-function mutations of ferroportin-1.

The invention also provides, “derivative” proteins which have been altered, for example by conjugation or complexing with other chemical moieties or by post-translational modification techniques as would be understood in the art. Such derivatives include amino acid deletions and/or additions to mutant ferroportin-1 proteins of the invention.

“Additions” of amino acids may include fusion of a mutant ferroportin-1 protein of the invention or a fragment thereof with other polypeptides or proteins. The other protein may, by way of example, assist in the purification of the protein. For instance, these include a polyhistidine tag, maltose binding protein (MBP), green fluorescent protein (GFP), Protein A or glutathione S-transferase (GST). Other additions include “epitope tags” such as FLAG and c-myc epitope tags.

Other derivatives contemplated by the invention include, but are not limited to, modification to side chains, incorporation of unnatural amino acids and/or their derivatives during peptide, polypeptide or protein synthesis and the use of crosslinkers and other methods which impose conformational constraints on the polypeptides, fragments and variants of the invention. Examples of side chain modifications contemplated by the present invention include modifications of amino groups such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with NaBH₄; reductive alkylation by reaction with an aldehyde followed by reduction with NaBH₄; and trinitrobenzylation of amino groups with 2,4,6-trinitrobenzene sulphonic acid (TNBS).

The carboxyl group may be modified by carbodiimide activation via O-acylisourea formation followed by subsequent derivitization, by way of example, to a corresponding amide.

The guanidine group of arginine residues may be modified by formation of heterocyclic condensation products with reagents such as 2,3-butanedione, phenylglyoxal and glyoxal.

Sulphydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulphides with other thiol compounds; reaction with maleimide, maleic anhydride or other substituted maleimide; carboxymethylation with iodoacetic acid or iodoacetamide; and carbamoylation with cyanate at alkaline pH.

Tryptophan residues may be modified, for example, by alkylation of the indole ring with 2-hydroxy-5-nitrobenzyl bromide or sulphonyl halides or by oxidation with N-bromosuccinimide.

Tyrosine residues may be modified by nitration with tetranitromethane to form a 3-nitrotyrosine derivative.

The imidazole ring of a histidine residue may be modified by N-carbethoxylation with diethylpyrocarbonate or by alkylation with iodoacetic acid derivatives.

Examples of incorporating unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids.

The invention also provides a recombinant mutant ferroportin-1 proteins of the invention (inclusive of fragments and derivatives) which may be prepared by any suitable procedure known to those of skill in the art.

For example, the recombinant protein may be prepared by a procedure including the steps of:

(i) preparing an expression construct which comprises an isolated nucleic acid according to FIG. 6 (SEQ. ID NO: 4), or at least a protein coding portion thereof, operably-linked to one or more regulatory nucleotide sequences in an expression vector;

(ii) transfecting or transforming a suitable host cell with the expression construct; and

(iii) expressing the recombinant protein in said host cell.

An “expression vector” may be either a self-replicating extra-chromosomal vector such as a plasmid, or a vector that integrates into a host genome.

By “operably-linked” is meant that said regulatory nucleotide sequence(s) is/are positioned relative to the recombinant nucleic acid of the invention to initiate, regulate or otherwise control transcription.

Regulatory nucleotide sequences will generally be appropriate for the host cell used for expression. Numerous types of appropriate expression vectors and suitable regulatory sequences are known in the art for a variety of host cells.

Regulatory nucleotide sequences present in the expression vector (such as an enhancer, promoter, splice donor/acceptor signals, terminator and polyadenylation sequences) that will facilitate expression of the mutant ferroportin-1 protein. Selectable markers are also useful whether for the purposes of selection of transformed bacteria (such as bla, kanR and tetR) or transformed mammalian cells (such as hygromycin, G418 and puromycin).

Both constitutive and inducible promoters may be useful for expression of the proteins of the invention. Examples of inducible promoters are metallothionine-inducible and tetracycline-repressible systems as are well known in the art.

In a preferred embodiment, the expression vector contains a selectable marker gene to allow the selection of transformed host cells.

Suitable host cells for expression may be prokaryotic or eukaryotic, such as Escherichia coli (DH5α for example), yeast cells, Sf9 cells utilized with a baculovirus expression system, CHO cells, COS, CV-1 and 293 cells, without limitation thereto.

The expression vector may also include a fusion partner (typically provided by the expression vector) so that the recombinant polypeptide of the invention is expressed as a fusion polypeptide with said fusion partner. The main advantage of fusion partners is that they assist identification and/or purification of said fusion polypeptide.

Well known examples of fusion partners include, but are not limited to, glutathione-S-transferase (GST), Fc portion of human IgG, maltose binding protein (MBP) and hexahistidine (HIS₆), which are particularly useful for isolation of the fusion polypeptide by affinity chromatography. For the purposes of fusion polypeptide purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system.

In some cases, the fusion partners also have protease cleavage sites, such as for Factor Xₐ or Thrombin, which allow the relevant protease to partially digest the fusion polypeptide of the invention and thereby liberate the recombinant polypeptide of the invention therefrom. The liberated polypeptide can then be isolated from the fusion partner by subsequent chromatographic separation.

The recombinant ferroportin-1 mutant protein may be conveniently prepared by a person skilled in the art using standard protocols as for example described in Sambrook et al., MOLECULAR CLONING. A Laboratory Manual (Cold Spring Harbor Press, 1989), incorporated herein by reference, in particular Sections 16 and 17; CURRENT PROTOCOLS IN MOLECULAR BIOLOGY Eds. Ausubel et al., (John Wiley & Sons, Inc. 1995-1999), incorporated herein by reference, in particular Chapters 10 and 16; and CURRENT PROTOCOLS IN PROTEIN SCIENCE Eds. Coligan et al., (John Wiley & Sons, Inc. 1995-1999) which is incorporated by reference herein, in particular Chapters 1, 5 and 6.

Antibodies

The invention also provides an antibody that binds an isolated mutant ferroportin-1 protein of the invention or a fragment thereof (such as an immunogenic peptide).

Preferably, an antibody of the invention binds said isolated mutant ferroportin-1 protein but does not bind, or binds with substantially lower affinity, to a corresponding wild-type ferroportin-1 protein.

Antibodies of the invention may be polyclonal or monoclonal. Well-known protocols applicable to antibody production, purification and use may be found, for example, in Chapter 2 of Coligan et al., CURRENT PROTOCOLS IN IMMUNOLOGY (John Wiley & Sons NY, 1995-2001) and Harlow, E. & Lane, D. Antibodies: A Laboratory Manual, Cold Spring Harbor, Cold Spring Harbor Laboratory, 1988, which are both herein incorporated by reference.

Polyclonal antibodies of the invention may be prepared, for example, by injecting a mutant ferroportin-1 protein of the invention, or a peptide lacking one or all of valines 160-162, into a production species, which may include mice or rabbits, to obtain polyclonal antisera. Methods of producing polyclonal antibodies are well known to those skilled in the art. Exemplary protocols which may be used are described for example in Coligan et al., CURRENT PROTOCOLS IN IMMUNOLOGY, supra, and in Harlow & Lane, 1988, supra.

In lieu of the polyclonal antisera obtained in the production species, monoclonal antibodies may be produced using the standard method as for example, described in an article by Köhler & Milstein, 1975, Nature 256, 495, which is herein incorporated by reference, or by more recent modifications thereof as for example, described in Coligan et al., CURRENT PROTOCOLS IN IMMUNOLOGY, supra by immortalizing spleen or other antibody producing cells derived from a production species which has been inoculated with one or more of the mutant proteins of the invention.

The invention also includes within its scope antibodies which comprise Fc or Fab fragments of the polyclonal or monoclonal antibodies referred to above. Alternatively, the antibodies may comprise single chain Fv antibodies (scFvs) against the mutant proteins of the invention. Such scFvs may be prepared, for example, in accordance with the methods described respectively in U.S. Pat. No. 5,091,513, European Patent No 239,400 or the article by Winter & Milstein, 1991, Nature 349, 293, which are incorporated herein by reference.

Labels may be associated with an antibody of the invention, or antibody fragment, as follows:

(A) direct attachment of the label to the antibody or antibody fragment;

(B) indirect attachment of the label to the antibody or antibody fragment; i.e., attachment of the label to another assay reagent which subsequently binds to the antibody or antibody fragment; and

(C) attachment to a subsequent reaction product of the antibody or antibody fragment.

The label may be selected from a group including a chromogen, a catalyst, an enzyme, a fluorophore, a chemiluminescent molecule, a lanthanide ion such as Europium (Eu³⁴), a radioisotope and a direct visual label. In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes useful as labels is disclosed in United States Patent Specifications U.S. Pat. No. 4,366,241, U.S. Pat. No. 4,843,000, and U.S. Pat. No. 4,849,338, all of which are herein incorporated by reference. Enzyme labels useful in the present invention include, for example, alkaline phosphatase, horseradish peroxidase, luciferase, O-galactosidase, glucose oxidase, lysozyme, malate dehydrogenase and the like. The enzyme label may be used alone or in combination with a second enzyme in solution.

The fluorophore may, for example, be fluorescein isothiocyanate (FITC), tetramethylrhodamine isothiocyanate (TRITC), allophycocyanin (APC), Texas Red, Cy5, Cy3, or R-Phycoerytbrin (RPE) as are well known in the art.

Isolated Nucleic Acids

The invention provides an isolated nucleic acid that encodes a mutant ferroportin-1 protein of the invention.

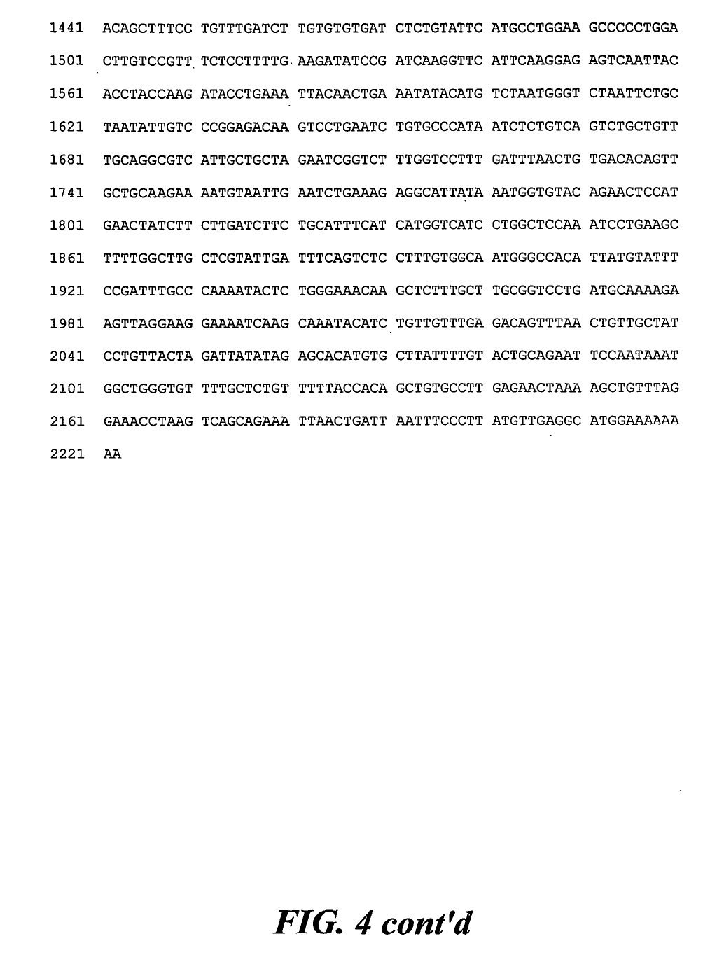

A mutant ferroportin-1 nucleic acid sequence of the invention is shown in FIG. 6 (SEQ ID NO: 4) and a wild-type ferroportin-1 nucleic acid sequence is shown in FIG. 4 (SEQ ID NO: 3).

In preferred embodiments, the invention provides fragments of the isolated nucleic acid of SEQ ID NO: 4 including the protein-coding region of SEQ ID NO: 4 and the region corresponding to exon 5 of SEQ ID NO: 4.

It will also be appreciated that the invention includes within its scope variations in nucleotide sequences of the invention on account of degeneracy in the genetic code.

Furthermore, the invention includes nucleotide sequences where codon sequences are optimized on the basis of preferred usage in a particular organism. These optimized sequences are particularly useful when a high level of encoded protein expression is required in said particular organism.

The term “nucleic acid” as used herein designates single-or double-stranded mRNA, RNA, cRNA and DNA inclusive of cDNA and genomic DNA.

A “polynucleotide” is a nucleic acid having eighty (80) or more contiguous nucleotides, while an “oligonucleotide” has less than eighty (80) contiguous nucleotides.

A “probe” may be a single or double-stranded oligonucleotide or polynucleotide, suitably labelled for the purpose of detecting complementary sequences in Northern or Southern blotting, for example.

A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™.

“Hybridize, hybridization, anneal and annealing” are used herein to denote the pairing of at least partly complementary nucleotide sequences to produce a DNA-DNA, RNA-RNA or DNA-RNA hybrid. Hybrid sequences comprising complementary nucleotide sequences occur through base-pairing between complementary purines and pyrimidines as are well known in the art.

In this regard, it will be appreciated that modified purines (for example, inosine, methylinosine and methyladenosine) and modified pyrimidines (for example, thiouridine and methylcytosine) may also engage in base pairing.

The higher the level of stringency during hybridization or annealing, the higher the degree of complementarity between the hybridized or annealed nucleic acid strands.

“Stringency” as used herein, refers to temperature and ionic strength conditions, and presence or absence of certain organic solvents and/or detergents during hybridisation.

“Stringent conditions” designates those conditions under which only nucleic acid having a high frequency of complementary bases will hybridize.

By way of example, high stringency conditions include and encompass:-

(i) from at least about 31% v/v to at least about 50% v/v formamide and from at least about 0.01 M to at least about 0.15 M salt for hybridisation at 42° C., and at least about 0.01 M to at least about 0.15 M salt for washing at 42° C.;

(ii) 1% BSA, 1 mM EDTA, 0.5 M NaHPO₄ (pH 7.2), 7% SDS for hybridization at 65° C., and (a) 0.1×SSC, 0.1% SDS; or (b) 0.5% BSA, 1 mM EDTA, 40 mM NaHPO₄ (pH 7.2), 1% SDS for washing at a temperature in excess of 65° C. for about one hour; and

(iii) 0.2×SSC, 0.1% SDS for washing at or above 68° C. for about 20 minutes.

In general, washing is carried out at Tₘ=69.3+0.41 (G+C) % -12° C. In general, the Tₘ of a duplex DNA decreases by about 1° C. with every increase of 1% in the number of mismatched bases.

Notwithstanding the above, stringent conditions are well known in the art, such as described in Chapters 2.9 and 2.10 of. Ausubel et al., supra, which are herein incorporated by reference.

The foregoing discussion is also relevant to detection and isolation of nucleic acids that are homologous to the isolated ferroportin-1 nucleic acid of the invention.

For example, the invention contemplates orthologous ferroportin-1 nucleic acid mutants isolated from mammals other than humans that may be useful in veterinary diagnosis of iron overload. For example, the invention contemplates mutant ferroportin-1 proteins isolated from laboratory species such as mice and rats, that may be useful in structure-function analysis of this mutant.

In one embodiment, complementary nucleotide sequences are identified by blotting techniques that include a step whereby nucleotides are immobilized on a matrix (preferably a synthetic membrane such as nitrocellulose), a hybridization step, and a detection step. Southern blotting is used to identify a complementary DNA sequence; northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al., supra, at pages 2.9.1 through 2.9.20. According to such methods, Southern blotting involves separating DNA molecules according to size by gel electrophoresis, transferring the size-separated DNA to a synthetic membrane, and hybridizing the membrane bound DNA to a complementary nucleotide sequence.

In dot blotting and slot blotting, DNA samples are directly applied to a synthetic membrane prior to hybridization as above. An alternative blotting step is used when identifying complementary nucleic acids in a cDNA or genomic DNA library, such as through the process of plaque or colony hybridization. Other typical examples of this procedure is described in Chapters 8-12 of Sambrook et al., supra which are herein incorpoated by reference.

Methods for detecting labeled nucleic acids hybridized to an immobilized nucleic acid are well known to practitioners in the art. Such methods include autoradiography, chemiluminescent, fluorescent and colorimetric detection.

Isolation of homologous ferroportin-1 mutant nucleic acids may also be readily performed using a nucleic acid sequence amplification technique.

Nucleic acid amplification techniques are well known to the skilled addressee, and include polymerase chain reaction (PCR) and ligase chain reaction (LCR) as for example described in Chapter 15 of Ausubel et al. supra, which is incorporated herein by reference; strand displacement amplification (SDA) as for example described in U.S. Pat. No. 5,422,252 which is incorporated herein by reference; rolling circle replication (RCR) as for example described in Liu et al., 1996, J. Am. Chem. Soc. 118, 1587 and International application WO 92/01813 and Lizardi et al., in International Application WO 97/19193, which are incorporated herein by reference; nucleic acid sequence-based amplification (NASBA) as for example described by Sooknanan et al., 1994, Biotechniques 17, 1077, which is incorporated herein by reference; and Q-β replicase amplification as for example described by Tyagi et al., 1996, Proc. Natl. Acad. Sci. USA 93, 5395, which is incorporated herein by reference.

Suitably, primers useful with nucleic acid sequence amplification techniques are capable of annealing to one or the other strands of a double-stranded nucleic acid under annealing conditions typically used for amplification. In the case of degenerate primers, sequence differences between the primer and the nucleic acid sequence are introduced so as to account for possible sequence variation, such as due to degeneracy in homologous coding sequences.

A preferred nucleic acid sequence amplification technique is PCR.

As used herein, an “amplification product” refers to a nucleic acid product generated by nucleic acid amplification techniques.

Mutation Detection

It will be well understood by the skilled person that detection of a mutant ferroportin-1 nucleic acid of the invention may be performed using any of a variety of techniques such as direct sequencing of genomic DNA or PCR amplification products, PCR-RFLP analysis, fluorescence-based melt curve analysis, Single Strand Conformational Polymorphism (SSCP) analysis, Denaturing Gradient Gel Electrophoresis (DGGE) or Denaturing High Performance Liquid Chromatography (DHPLC).

Detection may be performed using fragments of SEQ ID NO: 4 such as a fragment corresponding to exon 5 or at least a portion thereof that normally encodes valine 160, 161 or 162.

Preferably, said mutant ferroportin-1 nucleic acid of the invention or fragment thereof is produced by nucleic acid sequence amplification such as PCR.

In a preferred embodiment, mutation detection is performed by measuring melting temperature differences between a wild-type ferroportin-1 nucleic acid sequence and a mutant ferroportin-1 nucleic acid sequence of the invention, or respective fragments thereof, when hybridized to a fluorescently-labeled probe.

A preferred method uses melt curve analysis employing fluorochrome-labeled anchor and sensor probes, wherein the sensor probe forms base-pair mismatches when annealing to mutant DNA strands, but not to wild-type strands, resulting in a measurable Tm difference. A specific example of how melt curve analysis can be used for mutation detection according to the present invention is provided hereinafter.

Alternatively, fluorescent DNA-intercalating dyes (such as SYBR Green I™) can reveal the presence of these base-pair mismatches by virtue of their lower melting temperature (Tₘ) compared to fully complementary sequences A number of other examples of allele-specific melt curve analysis can be found, for example, in International Publication NO: WO97/46714.

An example of DHPLC applicable to mutation detection is also provided hereinafter.

DGGE also exploits Tₘ differences, but uses differential electrophoretic migration through gradient gels as a means of distinguishing subtle nucleotide sequence differences between alleles. Examples of DGGE methods can be found in Folde & Loskoot, 1994, and U.S. Pat. Nos. 5,045,450 and 5,190,856.

The invention also contemplates detection by microarrays that incorporate a mutant ferroportin-1 nucleic acid of the invention, or an oligonucleotide derived therefrom, which has a deletion of a codon encoding valine 162. Microarray, or nucleic acid array technology is well known in the art and described, for example, in detail in Unit 22, CURRENT PROTOCOLS IN MOLECULAR BIOLOGY Eds. Ausubel et al., (John Wiley & Sons, Inc. 1995-1999).

In another embodiment, a mutant ferroportin-1 protein is detected by an antibody that binds said mutant ferroportin-1 protein but does not bind, or has a substantially lower affinity for, a corresponding wild-type ferroportin-1 protein.

Suitable antibody-mediated detection systems are well known in the art and include ELISA, western blotting and immunohistochemistry. A detailed discussion of ELISA can be found in Unit 11.2, CURRENT PROTOCOLS IN MOLECULAR BIOLOGY Eds. Ausubel et al., (John Wiley & Sons, Inc. 1995-1999). Western blot, or immunoblot, is described in detail in Unit 10.8, CURRENT PROTOCOLS IN MOLECULAR BIOLOGY Eds. Ausubel et al., (John Wiley & Sons, Inc. 1995-1999).

Treatment of Iron Overload Deficiency

The invention also contemplates treating mammals, preferably humans with an iron overload disorder, preferably haemochromatosis, said method including the step of complementing a mutant allele in a mammal encoding the loss of function mutant ferroportin-1 protein.

One method of treatment of iron overload disease is gene therapy. Gene therapy constructs for complementing a ferroportin-1 loss of function mutation are also within the scope of the invention.

Gene therapy vectors may comprise an isolated nucleic acid that encodes a wild-type ferroportin-1 protein. Said gene therapy construct may be administered to an individual, preferably a human, suffering from iron overload disease due to a loss of function mutation in a ferroportin-1 gene. Suitable expression of said wild-type ferroportin-1 protein, preferably in the liver, would overcome haploinsufficiency in a heterozygous mutant individual, for example.

Gene therapy vectors may be expression vectors such as viral vectors such as vaccinia. The latter also include adenovirus and adenovirus-associated viruses (AAV) such as described in Braun-Falco et al.,1999, Gene Ther. 6, 432, retroviral and lentiviral vectors such as described in Buchshacher et al., 2000, Blood 95, 2499 and vectors derived from herpes simplex virus and cytomegalovirus. A general overview of viral vectors useful in endocrine gene therapy is provided in Stone et al., 2000, J. Endocrinol. 164, 103.

Applicable methods for the treatment of iron overload disease are the use of pharmaceutical compositions.

In one embodiment, the pharmaceutical composition comprises one or more cells engineered to express a wild-type ferroportin-1 protein and thereby recover iron homeostasis in an individual carrying a mutant ferroportin-1 allele.

A suitable cell type for this purpose would be a cell that is capable of regenerating liver such as a hepatocyte or a Kupffer cell. Liver cell transplantation for the treatment of acute, chronic or genetic liver insufficiency or disease is reviewed by Allen and Soriano, 2001, J. Lab. Clin. Med., 138,298-312. A general overview of liver-directed gene therapy and methods of gene transfer to the liver for a broad range of acquired and inherited hepatic disorders is provided in Ghosh et al., 2000, Journal of Hepatology, 32, 238-252.

In another embodiment, the pharmaceutical composition comprises a gene therapy construct as hereinbefore described.

Suitably, the pharmaceutical composition comprises an appropriate pharmaceutically-acceptable carrier, diluent or excipient.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991) which is incorporated herein by reference.

Any safe route of administration may be employed for providing a patient with the composition of the invention. For example, oral, rectal, parenteral, sublingual, buccal, intravenous, intra-articular, intramuscular, intra-dermal, subcutaneous, inhalational, intraocular, intraperitoneal, intracerebroventricular, transdermal and the like may be employed. Intra-muscular and subcutaneous injection is appropriate, for example, for administration of immunogenic compositions, vaccines and DNA vaccines.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Pharmaceutical compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

The above compositions may be administered in a manner compatible with the dosage formulation, and in such amount as is pharmaceutically-effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

So that the invention may be readily understood and put into practical effect, the skilled addressee is referred to the following non-limiting examples.

### EXAMPLES

### Example 1

Patients and Methods

Patients

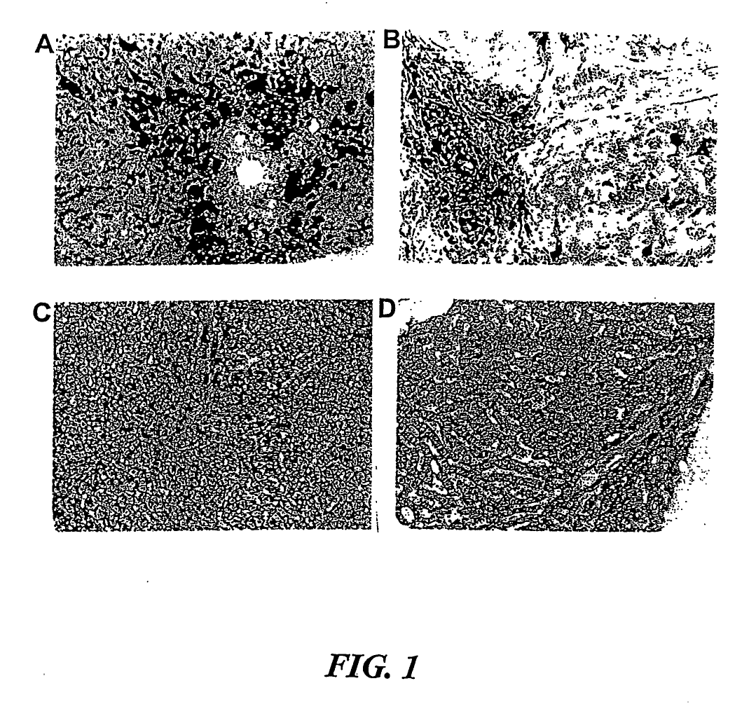

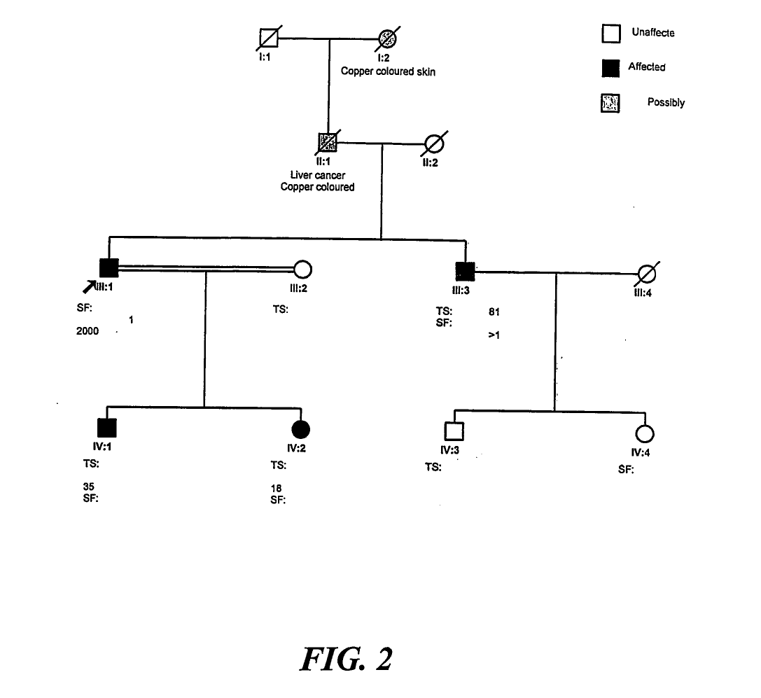

The proband presented in 1988 at age 56 years with thrombocytopenia. He also had hepatomegaly and skin pigmentation. Serum ferritin concentration was 12,000 μg/l. Liver histology showed portal fibrosis and Perls' stain grade 4. Iron was present in both hepatocytes and Kupffer cells (FIG. 1A). The hepatic iron concentration (HIC) was 475 μmoles/ gram dry weight and the hepatic iron index (HII) was 8.3 (HIC divided by age in years). He was treated by venesection and required the removal of 80 g of iron to return his serum ferritin concentration to normal levels. Family screening of the proband revealed 3 other affected relatives (Table 1, FIG. 2). The son and daughter (IV:1 and IV:2) were diagnosed at ages 20 and 19 years respectively. Both had raised serum ferritin concentrations, but normal transferrin saturations (Table 1). Liver biopsy revealed mild fibrosis in the son. The brother of the proband (III:3) presented at age 73 years. He had a raised serum ferritin concentration and transferrin saturation (Table 1). Liver biopsy sections from all affected family members are shown in FIG. 1. All have prominent iron staining in Kupffer cells. None of the affected family members had any problems tolerating weekly venesection therapy, however, haemoglobin levels were not monitored. There was no family history of haemochromatosis in this pedigree, however, the father of the proband (II:1) died of liver cancer and both the father (II:1) and grandmother (II:2) were reported as having copper coloured skin.

Controls

A control group comprising 103 Australian individuals was studied to determine the frequency of a novel ferroportin-1 mutation and exclude it as a common polymorphism.

Molecular Studies

DNA was prepared from peripheral blood using standard methods.¹⁸ The HFE mutations C282Y, H63D and S65C were screened for in all affected family members as described.^{19,20} The Y250X mutation of TfR2 was screened for by PCR and restriction endonuclease digestion with Bfa I. The entire coding region and splice sites of HFE were sequenced in the proband as described.²⁰

The entire coding region and splice sites of the ferroportin-1 gene were amplified from the proband using the primer pairs shown in Table 2. PCR was performed using 25 pmol of each primer and Promega PCR mastermix (Promega, Annandale, NSW, Australia) in a 50 μL final reaction volume in a Perkin Elmer DNA Thermal Cycler (Perkin Ehner, Wellesley, Mass.). PCR products were purified using the QIAquick PCR purification kit (QIAGEN Pty Ltd, Clifton Hill, VIC, Australia). Cycle sequencing was performed using ABI PRISM Big Dye Terminators (Applied Biosystems, Foster City, Calif.) in a PTC-100 Programmable Thermal Controller (MJ Research Inc., Watertown, Mass.). Sequencing products were run in an ABI PRISM 377 DNA Sequencer (Applied Biosystems, Foster City, Calif.).

PCR products of exon 5 from the affected and unaffected family members were also tested by denaturing high performance liquid chromatography (DHPLC) using a Varian ProStar Helix System (Varian Chromatography Systems, Walnut Creek, Calif.). Four analysis temperatures were initially used: 55, 56, 57 and 58° C. Heteroduplexes were seen at all temperatures in affected family members heterozygous for 485_{—}487delTTG. A temperature of 56° C. was deemed to be optimal for analysis. This corresponded with the optimal temperature recommended by the DHPLC Melt Program (http://insertion.stanford.edu/melt.html). Subsequently 103 control samples were analysed to determine the frequency of this mutation.

Results

In an effort to identify the genetic defect responsible for iron overload in this family the present inventors screened all affected family members for the HFE mutations C282Y, H63D and S65C and the TfR2 mutation Y250X. The brother and daughter of the proband (III:3 and IV:2) were heterozygous for H63D. None of the other HFE or TfR2 mutations were detected. Sequencing of the entire HFE coding region and splice sites in the proband (III:1) did not detect any other pathogenic mutations.

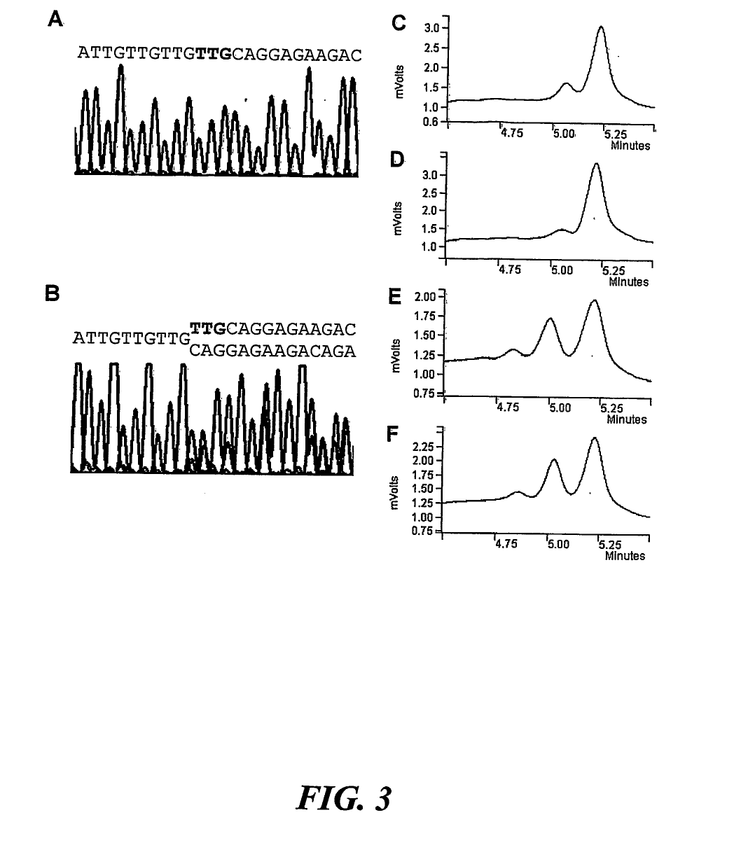

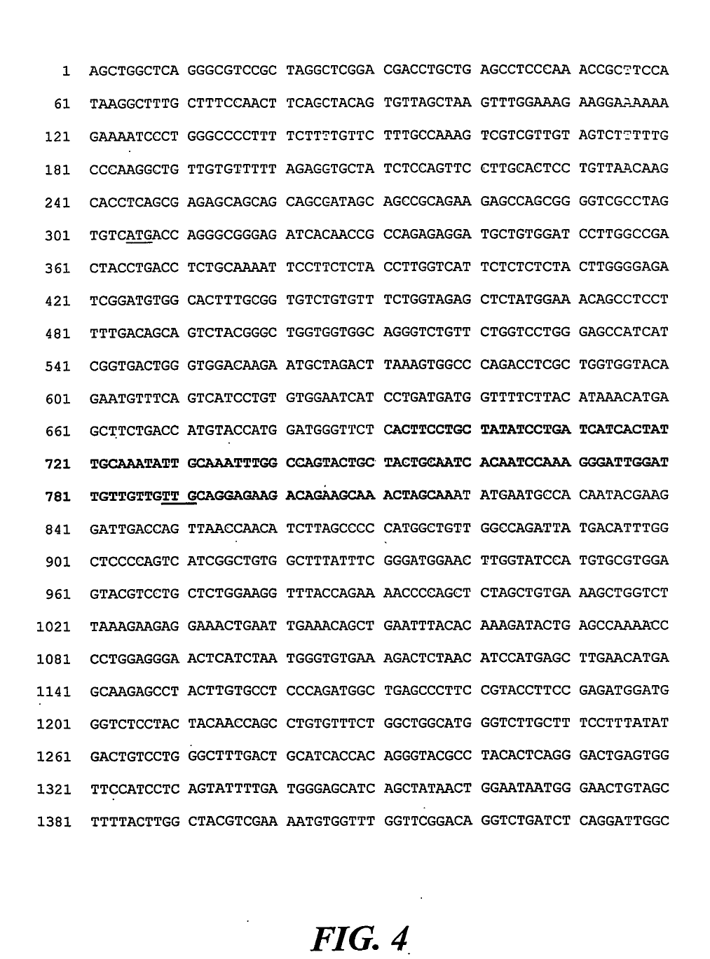

The entire coding region and splice sites of ferroportin-1 were sequenced in the proband. A heterozygous deletion of 3 base pairs (TTG) was detected in exon 5 (FIG. 3B). Sequencing of exon 5 in all family members showed that the 3 base pair deletion was present in all affected members, but was absent in all unaffected members (FIG. 3A). This 3 base pair deletion (485_{—}487delTTG) would predict the loss of a valine from the amino acid sequence of the ferroportin-1 protein (V162del). This mutation deletes one of 3 valine residues which are highly conserved across species and are predicted to reside in a 29 amino acid loop between transmembrane helices 3 and 4 of the ferroportin-1 protein.¹⁴

PCR fragments of exon 5 were further analyzed by denaturing HPLC. DHPLC analysis could detect the 3 base pair deletion at the four melting temperature profiles used. The presence of the deletion in all affected and absence in all unaffected family members was confirmed by this method (FIG. 3C-F). To confirm that this deletion was not a common polymorphism and to estimate its frequency, a control group comprising 103 individuals was analysed. The mutation was not detected in any of the controls studied.

Discussion

The present inventors describe a new mutation in the human ferroportin-1 gene in a family with autosomal dominant haemochromatosis. The phenotype of iron overload in this family differs from HFE related haemochromatosis. Serum ferritin levels in these patients are elevated early in the course of disease, whereas transferrin saturations are not elevated until later in life. Iron accumulation in the liver has a different pattern to classical HFE related haemochromatosis. Iron is seen predominantly in Kupffer cells in these patients.

This is the third reported mutation in ferroportin-1 associated with autosomal dominant haemochromatosis. It is unlikely that all 3 mutations cause a gain in function of a membrane transport protein as proposed for the N144H mutation.¹² The present inventors propose that heterozygosity for these mutations cause loss of function leading to haploinsufficiency for the ferroportin-1 protein and impaired iron recycling by reticuloendothelial (RE) macrophages. The flux of iron through the RE macrophages far exceeds the flux of iron through the duodenal mucosa.²¹ Therefore, we would suggest that haploinsufficiency for ferroportin-1 would be more limiting to iron transport in RE cells than in duodenal enterocytes. The large amount of iron taken up by phagocytosis of senescent red blood cells would not be able to be released to the circulation as readily. This would lead to a build up of iron in RE cells and result in low serum iron levels, which would cause subsequent signalling to the duodenal enterocytes to absorb more iron.

It is interesting to note that zebrafish weissherbst (weh) mutants, which are homozygous for mutations in ferroportin-1 have a contrasting phenotype.¹⁴ Mutant embryos have hypochromic anaemia due to iron deficiency and die by embryonic day 14. The iron deficiency of mutant embryos is due to a defect in iron transport from the yolk sac to the embryo.¹⁴ No information was given on heterozygotes for the weh mutations. It would be interesting to study fish heterozygous for ferroportin-1 mutations to see whether iron homeostasis is impaired and if they develop iron overload in adulthood.

The finding of a new mutation in ferroportin-1 in non HFE related haemochromatosis lends further support for the key role that this gene plays in body iron homeostasis. In a clinical setting mutation analysis of ferroportin-1 will be useful in patients with non HFE related iron overload, especially in families which appear to have autosomal dominant inheritance.

### Example 2

Detection of TTG (Val162del) Deletion in Exon 5 of Ferroportin-1 by Real Time PCR and Melting Curve Analysis

Template Preparation

Human genomic DNA was purified from buffy coat. Fresh blood samples were centrifuged and the buffy coat layer was removed to saline. After lysis and removal of erythrocytes the leucocytes were lysed in SDS and proteinase K at 56° C. overnight. Genomic DNA was extracted using phenol:chloroform and then precipitated in ethanol and resuspended in TE.

To make a synthetic mutant control, exon 5 of ferroportin-1 was amplified from mutant human genomic DNA in a total of 50 μl, containing 1× Reaction Buffer II (Applied Biosystems, Australia), 3 mM MgCl₂, 100 nM of both primers (IRG5F and IRG5R), 200 μM dNTPs and 1.25 U AmpliTaq Gold (Applied Biosystems, Australia). The cycling conditions were 7 minutes at 95° C. and then 45 cycles consisting of 94° for 30 seconds, 55° for 30 seconds and 72° for 30 seconds and a final 72° step for 10 minutes. The resulting PCR fragment was cloned into pGEM-T (Promega, Australia).

Primer sequences:

| | |
|---|---|
| IRG5F | |
| CTG CTA TAT CCT GAT CAT CAC TAT T | (SEQ ID NO: 5) |
| IRG5R | |
| GAA AGC CAA ATT ACT TGC TAG TT | (SEQ ID NO: 6) |

Exon 5 primer mix: 2 μM IRG5-F primer and 5 μM IRG5-R primer
100 nM and 250 nM final concentration in mix respectively

Probe sequences:

| | | |
|---|---|---|
| IRG5-V162 | _{LC Red640}GAT TGT TGT TGC AGG AGA AGA CAG A | (SEQ ID NO: 7) |
| IRG5-AnF | _{FITC}ACT GCT ACT GCA ATC ACA ATC CAA AGG GA_{FITC} | (SEQ ID NO: 8) |

The 5° FITC is not essential for this assay. The 5° FITC allows detection of a second mutation at the 5′ end of exon 5, in conjunction with a second sensor probe labelled at the 3′ end with LC-Red 640. All exon 5 probes bind to the reverse DNA strand.

Methods

The final concentration of the probe in the mix was 100 nM.

The PCR reaction mixture was a total volume of 10 μl comprising 1 μl template DNA and:

5.7 μL H₂O

0.5 μL exon 5 primer mix (2:5, IRG5-F:IRG5-R)

0.5 μL IRG5-V162 probe

0.5 μL IRG5-AnF probe

0.8 μL MgCl₂

1.0 μL Master mix

Samples were placed in glass capillaries (Roche) and run in a Roche LightCycler.

Cycling conditions:
All ramp speeds 20°/sec except during continuous acquisition in melt.
95° 480 sec (8 min)

| 95°, 4 sec | | |
|---|---|---|
| 55°, 2 sec | | |
| (single acquisition) | {close oversize brace} | 55 cycles Acquisition optional during |
| 55°, 6 sec | | amplification - faster without acquisition |

72°, 10 sec
Melt Conditions:
95°, 20 sec
40°, 0 sec

Ramp to 85° at 0.3°/sec with continuous acquisition
40°, 30 sec

Real Time PCR

The ferroportin-1 nucleic acid mutation was detected using a single-tube PCR (Roche Diagnostics, Australia) in the LightCycler, a combined thermocycler and fluorimeter (Roche, Australia).

A set of primers was designed to amplify the sequence from exon 5 of the human ferroportin gene. The probes were designed as follows. IRG5-V162 was labelled at the 5′ end with LC 640 and spans the 3 nt deletion mutation at positions 485 to 487, with complete homology with the mutant sequence (that is, with TTG missing in respect to the wildtype sequence). IRG5-AnF was labelled with FITC at the 3′ end and binds with high melting temperature (Tm) upstream of the mutation. The probes were designed to have different Tms to allow genotype identification by melting curve analysis.

For the real time assay, each glass capillary (Roche) contained a 10 μl reaction mix including 1 μl template DNA, 3 mM MgCl₂, primer pairs at 100 nM for IRG5F and 250 nM for IRG5R (Sigma Genosys, Australia) and fluorescently labelled probes (IRG5-V162 and IRG5-AnF) at 100 nM (TIB-MOLBIOL, Germany). The capillaries were then capped, loaded into the LightCycler's carousel and spun in the carousel centrifuge at 800×g (Roche, Australia). Once loaded into the LightCycler, the reaction mixes were exposed to an 8 minute, 95° C. polymerase activation step, followed by 55 cycles consisting of 94° C. for 4 seconds, 55° C. for 8 seconds and 72° C. for 15 seconds. At the completion of the amplification phase, the amplicon was denatured at 95° C. for 20 seconds, then cooled rapidly to 40° C. and finally heated to 95° C. at 0.3° C./second with continuous monitoring of the fluorescent emissions by the LightCycler. These data were presented as the negative derivative of the fluorescence over time, resulting in a melt curve. The real-time PCR took approximately 60 minutes to complete.

Results

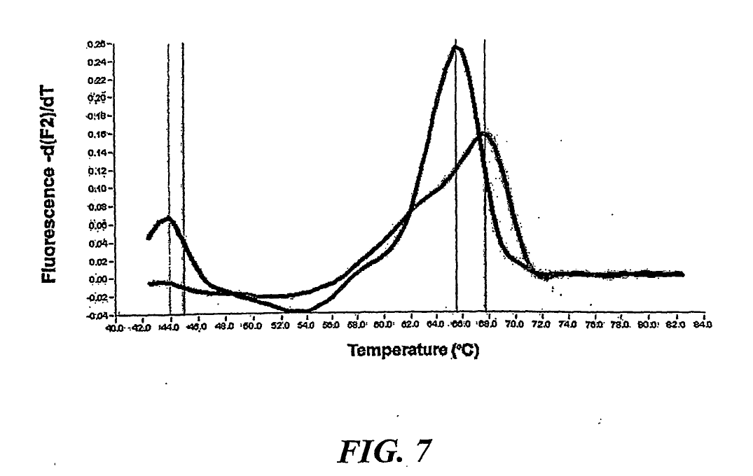

Using wild-type human genomic DNA and mutant DNA as purified plasmid containing the TTG deletion (val162del) mutation, the present inventors have shown distinct melting peaks for the wild-type and mutant alleles of this ferroportin-1 mutation. The melting peak for the wild-type allele occurs at 65.5° C. and the mutant melting peak occurs at 68° C. (FIG. 7). When patients carrying the mutant are tested, peaks for both wild-type and mutant alleles are expected if they are heterozygous for the mutation.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

TABLE 1

| | | | | Tf. | Serum | | | | Iron |
|---|---|---|---|---|---|---|---|---|---|
| Family | | Age at | HFE | sat. | ferritin | Hepatic | Perls' | | removed |
| member | Sex | dx (y) | genotype | (%) | (μg/l) | pathology | grade | HII | (g) |
| III:1 | M | 56 | HH/CC | | 12000 | Fibrosis | 4 | 8.3 | 80 |
| III:3 | M | 73 | HD/CC | 81 | >10000 | Fibrosis | 4 | — | Not yet |
| | | | | | | | | | deironed |
| IV:1 | M | 20 | HH/CC | 35 | 1768 | Mild | 3 | 5.2 | 17 |
| | | | | | | fibrosis | | | |
| IV:2 | F | 19 | HD/CC | 19 | 1182 | Normal | 2 | 3.5 | 3.25 |

TABLE 2

Size

| | | |
|---|---|---|
| Primer name | Sequence 5′ to 3′ | (bp) |

| | | |
|---|---|---|
| Exon 1 forward | CCCCGACTCGGTATAAGAGC | 504 |
| (SEQ ID NO: 9) | | |
| Exon 1 reverse | CACAGCAGAGCCACATTCC | |
| (SEQ ID NO: 10) | | |
| Exon 2 forward | TCATGTTCTAATGAGATCAAATGG | 257 |
| (SEQ ID NO: 11) | | |
| Exon 2 reverse | TGACAAAACTGGAAGTTGGC | |
| (SEQ ID NO: 12) | | |
| Exon 3 forward | ATGTAGCCAGGAAGTGCCC | 365 |
| (SEQ ID NO: 13) | | |
| Exon 3 reverse | TTCCCTGGTTGTTTCTCTCC | |
| (SEQ ID NO: 14) | | |
| Exon 4 forward | CAGAAAGGTTTTCTTTTTATCTGG | 369 |
| (SEQ ID NO: 15) | | |
| Exon 4 reverse | AATCAATATTAAAAGGTCACACTGG | |
| (SEQ ID NO: 16) | | |
| Exon 5 forward | TCCACCAAAGACTATTTTAAACTGC | 321 |
| (SEQ ID NO: 17) | | |
| Exon 5 reverse | ACCCAGAACAAAAATACAAGGC | |
| (SEQ ID NO: 18) | | |
| Exon 6 forward | AGGAATCTATACTCTTGGTTTACAGC | 471 |
| (SEQ ID NO: 19) | | |
| Exon 6 reverse | ATTTAACCTCATCTGGCCCC | |
| (SEQ ID NO: 20) | | |
| Exon 7 forward | TTGGGAAGGGGAATAGAAGG | 868 |
| (SEQ ID NO: 21) | | |
| Exon 7 reverse | TTTCGTAAGAGTGGATTTTGC | |
| (SEQ ID NO: 22) | | |
| Exon 8 forward | GGCAAGGCTATGGTATATTTAAGG | 559 |
| (SEQ ID NO: 23) | | |
| Exon 8 reverse | AACAGAGCAAAACACCCAGC | |
| (SEQ ID NO: 24) | | |

### References

1. Edwards C Q, Griffen L M, Goldgar D, Drummond C, Skolnick M H, Kushner J P. Prevalence of hemochromatosis among 11,065 presumably healthy blood donors. N Engl J Med. 1988;318:1355-1362.

2. Bothwell T H, Charlton R W, Motulsky A G. Hemochromatosis. In: Scriver C R, Beaudet A L, Sly W S, Valle D, eds. The Metabolic and Molecular Bases of Inherited Disease, 7th edition. New York, N.Y.: McGraw-Hill; 1995:2237-2269.

3. Feder J N, Gnirke A, Thomas W, et al. A novel MHC class I-like gene is mutated in patients with hereditary haemochromatosis. Nat Genet. 1996;13:399-408.

4. Piperno A, Sampietro M, Pietrangelo A, et al. Heterogeneity of hemochromatosis in Italy. Gastroenterology. 1998; 114:996-1002.

5. Camaschella C. Juvenile hemochromatosis. Baillieres Clin Gastroenterol. 1998;12:227-235.

6. Roetto A, Totaro A, Cazzola M et al. The juvenile hemochromatosis locus maps to chromosome lq. Am J Hum Genet. 1999;64:1388-1393.

7. Camascella C, Roetto A, Cali A, et al. The gene TfR2 is mutated in a new type of haemochromatosis mapping to 7q22. Nat Genet. 2000;25:14-15.

8. Roetto A, Totaro A, Pipemo A, et al. New mutations inactivating transferrin receptor 2 in hemochromatosis type 3. Blood. 2001;97:2555-2560.

9. Eason R J, Adams P C, Aston C E, Searle J. Familial iron overload with possible autosomal dominant inheritance. Aust NZ J Med. 1990;20:226-230.

10. Pietrangelo A, Montosi G, Totaro A. Hereditary hemochromatosis in adults without pathogenic mutations in the hemochromatosis gene. N Engl J Med. 1999;341 :725-732.

11. Dooley J S, Wallace D F, Walker A P. Familial adult haemochromatosis (HC) with normal HFE sequence [abstract]. J Hepatol. 1999;30 (suppl 1):160.

12. Njajou O T, Vaessen N, Joosse M, et al. A mutation in SLC11A3 is associated with autosomal dominant hemochromatosis. Nat Genet. 2001;28:213-214.

13. Montosi G, Donovan A, Totaro A, et al. Autosomal dominant hemochromatosis is associated with a mutation in the ferroportin (SLC11A3) gene. J Clin Invest. 2001;108:619-623.

14. Donovan A, Brownlie A, Zhou Y, et al. Positional cloning of zebrafish ferroportin1 identifies a conserved vertebrate iron exporter. Nature. 2000;403 :778-781.

15. McKie A T, Marciani P, Rolfs A, et al. A novel duodenal iron-regulated transporter, IREG1, implicated in the basolateral transfer of iron to the circulation. Mol Cell. 2000;5:299-309.

16. Abboud S, Haile D J. A novel mammalian iron-regulated protein involved in intracellular iron metabolism. J Biol Chem. 2000;275:19906-19912.

17. Kato J, Fujikawa K, Kanda M, et al. A mutation, in the iron-responsive element of H ferritin mRNA, causing autosomal dominant iron overload. A J Hum Genet. 2001;69:191-197.

18. Sambrook J, Russell D W. Molecular Cloning: a Laboratory Manual, 3rd edition. Cold Spring Harbor, N.Y.: Cold Spring Harbor Laboratory Press; 2001.

19. Worwood M, Shearman J D, Wallace D F, et al. A simple genetic test identifies 90% of UK patients with haemochromatosis. Gut; 1997:41:841-844.

20. Wallace D F, Dooley J S, Walker A P. A novel mutation of HFE explains the classical phenotype of genetic hemochromatosis in a C282Y heterozygote. Gastroenterology. 1999;116:1409-1412.

1. An isolated mutant ferroportin-1 protein wherein said protein has a deletion or non-conservative substitution of a valine residue selected from the group consisting of: valine 160, valine 161 and valine 162, with respect to the wild-type ferroportin-1 amino acid sequence of SEQ I) NO: 1.

2. The isolated mutant ferroportin-1 protein of claim 1 wherein said protein has a deletion of a valine residue selected from the group consisting of: valine 160, valine 161 and valine 162.

3. The isolated mutant ferroportin-1 protein of claim 2 wherein said protein has a deletion of a valine 162.

4. The isolated protein of claim 1 having the amino acid sequence set forth in SEQ ID NO: 2.

5. A fragment of the isolated protein of claim 4, encoded by at least a portion of exon 5 of SEQ ID NO: 4.

6. An isolated nucleic acid encoding the ferroportin-1 mutant protein of claim 1.

7. The isolated nucleic acid of claim 6 which has a nucleotide sequence having one or more deleted or non-synonymous nucleotides that normally encode a valine residue selected from the group consisting of: valine 160, valine 161 and valine 162.

8. The isolated nucleic acid of claim 7 which comprises a nucleotide sequence having one or more deleted nucleotides that normally encode valine 162.

9. A fragment of the isolated nucleic acid of claim 6 having at least a protein-encoding nucleotide sequence set forth in SEQ ID NO: 4.

10. A fragment of the isolated nucleic acid of claim 6 corresponding to at least a portion of exon 5 of the human ferroportin-1 gene.

11. An antibody that binds the isolated mutant ferroportin-1 protein of claim 1, or a fragment thereof, but does not bind a corresponding wild-type ferroportin-1 protein or fragment thereof.

12. An expression construct comprising an isolated nucleic acid according to claim 6, wherein said nucleic acid is operably-linked to one or more regulatory sequences in an expression vector.

13. A host cell transfected or transformed with the expression construct of claim 12.

14. A method of detecting a predisposition to an iron overload disorder, said method including the step of detecting an isolated mutant ferroportin-1 nucleic acid according to claim 6, or a fragment thereof, as an indication that an individual is predisposed to said iron overload disorder.

15. The method of claim 14 wherein said iron overload disorder is haemochromatosis.

16. The method of claim 14 wherein detection is performed by a PCR method.

17. The method of claim 16 wherein the PCR is real time fluorescent PCR.

18. The method of claim 14 wherein PCR amplification products corresponding to wild-type and loss-of-function ferroportin-1 nucleic acids respectively are identified by differential melting temperatures.

19. The method of claim 18 wherein differential melting temperatures are measured by using a fluorescent probe that differentially hybridises to a mutant ferroportin-1 nucleic acid compared to a wild-type ferroportin-1 nucleic acid.

20. The method of claim 18 wherein differential melting temperatures are measured by Denaturing HPLC.

21. A method of detecting a predisposition to an iron overload disorder, said method including the step of detecting a loss-of-function mutant ferroportin-1 protein according to claim 1, or a fragment thereof, as an indication that an individual is predisposed to said iron overload disorder.

22. The method of claim 21 wherein said iron overload disorder is haemochromatosis.

23. A method of treating an iron overload disorder, said method including the step of complementing a mutant allele in a mammal encoding the mutant ferroportin-1 protein of claim 1 in said mammal.

24. The method of claim 23 wherein the iron overload disorder is haemochromatosis.

25. The method of claim 23 wherein complementation is achieved by gene therapy.

26. The method of claim 24 wherein said mammal is a human.


SEQUENCE LISTING (24 sequences)

SEQ ID NO: 1 - protein, 571 aa, Homo sapiens
MTRAGDHNRQRGCCGSLADYLTSAKFLLYLGHSLSTWGDRMWHFAVSVFLVELYGNSLLLTAVYGLVVAGSVLVLGAIIGDWVDKNARLKVAQTSLVVQNVSVILCGIILMMVFLHKHELLTMYHGWVLTSCYILIITIANIANLASTATAITIQRDWIVVVAGEDRSKLANMNATIRRIDQLTNILAPMAVGQIMTFGSPVIGCGFISGWNLVSMCVEYVLLWKVYQKTPALAVKAGLKEEETELKQLNLHKDTEPKPLEGTHLMGVKDSNIHELEHEQEPTCASQMAEPFRTFRDGWVSYYNQPVFLAGMGLAFLYMTVLGFDCITTGYAYTQGLSGSILSILMGASAITGIMGTVAFTWLRRKCGLVRTGLISGLAQLSCLILCVISVFMPGSPLDLSVSPFEDIRSRFIQGESITPTKIPEITTEIYMSNGSNSANIVPETSPESVPIISVSLLFAGVIAARIGLWSFDLTVTQLLQENVIESERGIINGVQNSMNYLLDLLHFIMVILAPNPEAFGLLVLISVSFVAMGHIMYFRFAQNTLGNKLFACGPDAKEVRKENQANTSVV

SEQ ID NO: 2 - protein, 570 aa, Homo sapiens
MTRAGDHNRQRGCCGSLADYLTSAKFLLYLGHSLSTWGDRMWHFAVSVFLVELYGNSLLLTAVYGLVVAGSVLVLGAIIGDWVDKNARLKVAQTSLVVQNVSVILCGIILMMVFLHKHELLTMYHGWVLTSCYILIITIANIANLASTATAITIQRDWIVVAGEDRSKLANMNATIRRIDQLTNILAPMAVGQIMTFGSPVIGCGFISGWNLVSMCVEYVLLWKVYQKTPALAVKAGLKEEETELKQLNLHKDTEPKPLEGTHLMGVKDSNIHELEHEQEPTCASQMAEPFRTFRDGWVSYYNQPVFLAGMGLAFLYMTVLGFDCITTGYAYTQGLSGSILSILMGASAITGIMGTVAFTWLRRKCGLVRTGLISGLAQLSCLILCVISVFMPGSPLDLSVSPFEDIRSRFIQGESITPTKIPEITTEIYMSNGSNSANIVPETSPESVPIISVSLLFAGVIAARIGLWSFDLTVTQLLQENVIESERGIINGVQNSMNYLLDLLHFIMVILAPNPEAFGLLVLISVSFVAMGHIMYFRFAQNTLGNKLFACGPDAKEVRKENQANTSVV

SEQ ID NO: 3 - DNA, 2222 bp, Homo sapiens
agctggctcagggcgtccgctaggctcggacgacctgctgagcctcccaaaccgcttccataaggctttgctttccaacttcagctacagtgttagctaagtttggaaagaaggaaaaaagaaaatccctgggccccttttcttttgttctttgccaaagtcgtcgttgtagtctttttgcccaaggctgttgtgtttttagaggtgctatctccagttccttgcactcctgttaacaagcacctcagcgagagcagcagcagcgatagcagccgcagaagagccagcggggtcgcctagtgtcatgaccagggcgggagatcacaaccgccagagaggatgctgtggatccttggccgactacctgacctctgcaaaattccttctctaccttggtcattctctctctacttggggagatcggatgtggcactttgcggtgtctgtgtttctggtagagctctatggaaacagcctccttttgacagcagtctacgggctggtggtggcagggtctgttctggtcctgggagccatcatcggtgactgggtggacaagaatgctagacttaaagtggcccagacctcgctggtggtacagaatgtttcagtcatcctgtgtggaatcatcctgatgatggttttcttacataaacatgagcttctgaccatgtaccatggatgggttctcacttcctgctatatcctgatcatcactattgcaaatattgcaaatttggccagtactgctactgcaatcacaatccaaagggattggattgttgttgttgcaggagaagacagaagcaaactagcaaatatgaatgccacaatacgaaggattgaccagttaaccaacatcttagcccccatggctgttggccagattatgacatttggctccccagtcatcggctgtggctttatttcgggatggaacttggtatccatgtgcgtggagtacgtcctgctctggaaggtttaccagaaaaccccagctctagctgtgaaagctggtcttaaagaagaggaaactgaattgaaacagctgaatttacacaaagatactgagccaaaacccctggagggaactcatctaatgggtgtgaaagactctaacatccatgagcttgaacatgagcaagagcctacttgtgcctcccagatggctgagcccttccgtaccttccgagatggatgggtctcctactacaaccagcctgtgtttctggctggcatgggtcttgctttcctttatatgactgtcctgggctttgactgcatcaccacagggtacgcctacactcagggactgagtggttccatcctcagtattttgatgggagcatcagctataactggaataatgggaactgtagcttttacttggctacgtcgaaaatgtggtttggttcggacaggtctgatctcaggattggcacagctttcctgtttgatcttgtgtgtgatctctgtattcatgcctggaagccccctggacttgtccgtttctccttttgaagatatccgatcaaggttcattcaaggagagtcaattacacctaccaagatacctgaaattacaactgaaatatacatgtctaatgggtctaattctgctaatattgtcccggagacaagtcctgaatctgtgcccataatctctgtcagtctgctgtttgcaggcgtcattgctgctagaatcggtctttggtcctttgatttaactgtgacacagttgctgcaagaaaatgtaattgaatctgaaagaggcattataaatggtgtacagaactccatgaactatcttcttgatcttctgcatttcatcatggtcatcctggctccaaatcctgaagcttttggcttgctcgtattgatttcagtctcctttgtggcaatgggccacattatgtatttccgatttgcccaaaatactctgggaaacaagctctttgcttgcggtcctgatgcaaaagaagttaggaaggaaaatcaagcaaatacatctgttgtttgagacagtttaactgttgctatcctgttactagattatatagagcacatgtgcttattttgtactgcagaattccaataaatggctgggtgttttgctctgtttttaccacagctgtgccttgagaactaaaagctgtttaggaaacctaagtcagcagaaattaactgattaatttcccttatgttgaggcatggaaaaaaaa

SEQ ID NO: 4 - DNA, 2219 bp, Homo sapiens
agctggctcagggcgtccgctaggctcggacgacctgctgagcctcccaaaccgcttccataaggctttgctttccaacttcagctacagtgttagctaagtttggaaagaaggaaaaaagaaaatccctgggccccttttcttttgttctttgccaaagtcgtcgttgtagtctttttgcccaaggctgttgtgtttttagaggtgctatctccagttccttgcactcctgttaacaagcacctcagcgagagcagcagcagcgatagcagccgcagaagagccagcggggtcgcctagtgtcatgaccagggcgggagatcacaaccgccagagaggatgctgtggatccttggccgactacctgacctctgcaaaattccttctctaccttggtcattctctctctacttggggagatcggatgtggcactttgcggtgtctgtgtttctggtagagctctatggaaacagcctccttttgacagcagtctacgggctggtggtggcagggtctgttctggtcctgggagccatcatcggtgactgggtggacaagaatgctagacttaaagtggcccagacctcgctggtggtacagaatgtttcagtcatcctgtgtggaatcatcctgatgatggttttcttacataaacatgagcttctgaccatgtaccatggatgggttctcacttcctgctatatcctgatcatcactattgcaaatattgcaaatttggccagtactgctactgcaatcacaatccaaagggattggattgttgttgcaggagaagacagaagcaaactagcaaatatgaatgccacaatacgaaggattgaccagttaaccaacatcttagcccccatggctgttggccagattatgacatttggctccccagtcatcggctgtggctttatttcgggatggaacttggtatccatgtgcgtggagtacgtcctgctctggaaggtttaccagaaaaccccagctctagctgtgaaagctggtcttaaagaagaggaaactgaattgaaacagctgaatttacacaaagatactgagccaaaacccctggagggaactcatctaatgggtgtgaaagactctaacatccatgagcttgaacatgagcaagagcctacttgtgcctcccagatggctgagcccttccgtaccttccgagatggatgggtctcctactacaaccagcctgtgtttctggctggcatgggtcttgctttcctttatatgactgtcctgggctttgactgcatcaccacagggtacgcctacactcagggactgagtggttccatcctcagtattttgatgggagcatcagctataactggaataatgggaactgtagcttttacttggctacgtcgaaaatgtggtttggttcggacaggtctgatctcaggattggcacagctttcctgtttgatcttgtgtgtgatctctgtattcatgcctggaagccccctggacttgtccgtttctccttttgaagatatccgatcaaggttcattcaaggagagtcaattacacctaccaagatacctgaaattacaactgaaatatacatgtctaatgggtctaattctgctaatattgtcccggagacaagtcctgaatctgtgcccataatctctgtcagtctgctgtttgcaggcgtcattgctgctagaatcggtctttggtcctttgatttaactgtgacacagttgctgcaagaaaatgtaattgaatctgaaagaggcattataaatggtgtacagaactccatgaactatcttcttgatcttctgcatttcatcatggtcatcctggctccaaatcctgaagcttttggcttgctcgtattgatttcagtctcctttgtggcaatgggccacattatgtatttccgatttgcccaaaatactctgggaaacaagctctttgcttgcggtcctgatgcaaaagaagttaggaaggaaaatcaagcaaatacatctgttgtttgagacagtttaactgttgctatcctgttactagattatatagagcacatgtgcttattttgtactgcagaattccaataaatggctgggtgttttgctctgtttttaccacagctgtgccttgagaactaaaagctgtttaggaaacctaagtcagcagaaattaactgattaatttcccttatgttgaggcatggaaaaaaaa

SEQ ID NO: 5 - DNA, 25 bp, artificial sequence
  primer
ctgctatatcctgatcatcactatt

SEQ ID NO: 6 - DNA, 23 bp, artificial sequence
  primer
gaaagccaaattacttgctagtt

SEQ ID NO: 7 - DNA, 25 bp, Artificial Sequence
  probe
gattgttgttgcaggagaagacaga

SEQ ID NO: 8 - DNA, 29 bp, artificial sequence
  probe
actgctactgcaatcacaatccaaaggga

SEQ ID NO: 9 - DNA, 20 bp, artificial sequence
  primer
ccccgactcggtataagagc

SEQ ID NO: 10 - DNA, 19 bp, artificial sequence
  primer
cacagcagagccacattcc

SEQ ID NO: 11 - DNA, 24 bp, artificial sequence
  primer
tcatgttctaatgagatcaaatgg

SEQ ID NO: 12 - DNA, 20 bp, artificial sequence
  primer
tgacaaaactggaagttggc

SEQ ID NO: 13 - DNA, 19 bp, artificial sequence
  primer
atgtagccaggaagtgccc

SEQ ID NO: 14 - DNA, 20 bp, artificial sequence
  primer
ttccctggttgtttctctcc

SEQ ID NO: 15 - DNA, 24 bp, artificial sequence
  primer
cagaaaggttttctttttatctgg

SEQ ID NO: 16 - DNA, 25 bp, Artificial Sequence
  primer
aatcaatattaaaaggtcacactgg

SEQ ID NO: 17 - DNA, 25 bp, artificial sequence
  primer
tccaccaaagactattttaaactgc

SEQ ID NO: 18 - DNA, 22 bp, artificial sequence
  primer
acccagaacaaaaatacaaggc

SEQ ID NO: 19 - DNA, 26 bp, artificial sequence
  primer
aggaatctatactcttggtttacagc

SEQ ID NO: 20 - DNA, 20 bp, artificial sequence
  primer
atttaacctcatctggcccc

SEQ ID NO: 21 - DNA, 20 bp, artificial sequence
  primer
ttgggaaggggaatagaagg

SEQ ID NO: 22 - DNA, 21 bp, artificial sequence
  primer
tttcgtaagagtggattttgc

SEQ ID NO: 23 - DNA, 24 bp, artificial sequence
  primer
ggcaaggctatggtatatttaagg

SEQ ID NO: 24 - DNA, 20 bp, artificial sequence
  primer
aacagagcaaaacacccagc